(19) United States
(12) Patent Application Publication
(10) Pub. No.: US 2022/0047903 A1
(43) Pub. Date: Feb. 17, 2022

(54) Nasal Air FIltration Apparatus

(71) Applicants: Valerie Fournier, WINTERS, CA (US); Alejandro Garcia, WINTERS, CA (US)

(72) Inventors: Valerie Fournier, WINTERS, CA (US); Alejandro Garcia, WINTERS, CA (US)

(21) Appl. No.: 17/303,134

(22) Filed: May 20, 2021

Related U.S. Application Data
(60) Provisional application No. 63/101,838, filed on May 18, 2020.

(51) Int. Cl. A62B 23/06; A61M 15/08

(52) U.S. Cl. CPC A62B 23/06; A61M 15/085

(57) ABSTRACT

The invention pertains to the field of wearable air purifiers; specifically, filtration focused on air breathed in via the nose. A nasal air filtration apparatus comprised layered compressible inner seal, filtration material, internal malleable skeleton to allow for custom shaping, optional outer gasket, and attachment device along midline. Apparatus is flat in profile with exception of attachment device until end user modification, and has one straight side with the other side being a long arch resulting in a roughly semicircular appearance. Apparatus attaches on midline of nose to a nasal dilation device, straight side oriented towards users' upper lip, arched side along edges of nostrils. Apparatus is then molded by user to form an airtight seal around users' nose, providing filtration of inspired air.

### CROSS-REFERENCE TO RELATED APPLICATIONS

This application claims priority to, and to the benefit of, U.S. Provisional Patent Ser. No. 63/101,838 filed on May 20, 2020 incorporated herein by reference in its entirety. Apparatus is intended to work in conjunction with, however not limited to only working with, a nasal dilation device as described in U.S. provisional patent No. 62/890,961.

### STATEMENT REGARDING FEDERALLY SPONSORED RESEARCH OR DEVELOPMENT

Not applicable

### NOTICE OF MATERIAL SUBJECT TO COPYRIGHT PROTECTION

A portion of the material in this patent document may be subject to copyright protection under the copyright laws of the US and other countries. The owner of the copyright rights has no objection to the facsimile reproduction by anyone of the patent document or the patent disclosure, as it appears in the United States Patent and Trademark Office publicly available file or records, but otherwise reserves all copyright rights whatsoever. The copyright owner does not hereby waive any of its rights to have this patent document maintained in secrecy, including without limit its rights pursuant to 37 C.F.R. 1.14.

### BACKGROUND

### 1. Technical Field

The technology of this disclosure pertains generally to wearable air purifiers, and more specifically to a wearable air filter apparatus configured to cover the wearers' external nares (nostrils).

### 2. Background Discussion

Poor air quality is a health hazard which we are becoming increasingly aware of. Pollution, wildfire smoke, allergens, and infectious agents are all potentially dangerous when inhaled. Short term and long term medical conditions, some fatal, have been linked to inhaling particulates.

Inspiration (inhaling air) is done primarily via the nose in a normal human. The nasal passages are designed to humidify and adjust the temperature of the inspired air, resulting in better oxygen extraction. Even during mild to moderate exercise, nasal vs oral inspiration is beneficial and the volume of air moved is negligibly impacted by inspiration route.

Full coverage face masks do not seal properly with facial hair, tend to move or gap when the wearer speaks, tend to be hot and uncomfortable, and limit communication due to blocking listeners' view of the mouth. This communication limitation is even more pronounced for those with hearing impairments or in situations where ambient noise is significant. Therefore, there is a need for an apparatus which offers the wearer protection from inhaling particulates through the nose, which is not impacted by facial hair, facial movements, and allows unimpeded communication.

### BRIEF SUMMARY

This disclosure describes an external filter designed to be attached to (Refer to U.S. provisional patent No. 62/890,961) and provide filtration of air inhaled through the external nares (nostrils).

In one embodiment, the device comprises 2 major parts:

The gasket structure is bilaterally symmetrical, roughly triangular/semicircular in shape. It is constructed of rigid but malleable material coated in a medically safe flexible material (which may include, but is not limited to: silicone, biosafe rubber, biosafe foam) such that it forms an airtight seal at the opening of the nares. The internal structure of the gasket may be comprised of wires or flat, thin metal to best fit the contours of the face and provide a repeatable shape which the individual wearer may adjust. The outer material of the gasket will “sandwich” (or cover on both sides) the inner structure providing both comfort and a good seal between the wearer's skin and the gasket.

Filtration material may include, but is not limited to, paper, cloth, pressed fiber, spun melted fiber, or other permutations/combinations. Filtration material may also include additives such as essential oils, medications, or additional specialized ingredients (silver or carbon, as examples) which may provide additional benefits beyond the physical filtration of inspired air.

In one embodiment, along the midline of the gasket is an attachment clip made of a stiff material such as, but not limited to, plastic or metal. This allows the device to attach firmly to the (Refer to U.S. provisional patent No. 62/890,961) and remain in place that way. Other attachment methods such as magnets, adhesives hooks or other forms of clips may be used to produce additional security.

When the device is attached to the (Refer to U.S. provisional patent No. 62/890,961) it is held in place such that an airtight seal is formed with the nares upon inhalation. Attachment to (Refer to U.S. provisional patent No. 62/890,961) is paramount, as this device both dilates the nasal passages and allows the weight of the device to be supported.

Using the device (by attaching it to (Refer to U.S. provisional patent No. 62/890,961) and proper adjustment to fit the wearer) produces a seal which upon inspiration is airtight, driving inspired air through the filtration material included in the gasket portion and filtering particulates out. The filtration device may be worn singly or in conjunction with other facial masks as preferred by the wearer to provide additional filtration capability, resulting in reduction of inhaled particulates by the wearer.

Further aspects of the technology described herein will be brought out in the following portions of the specification, wherein the detailed description is for the purpose of fully disclosing preferred embodiments of the technology without placing limitations thereon.

### BRIEF DESCRIPTION OF THE SEVERAL VIEWS OF THE DRAWINGS

These and other features and advantages of the present invention will be better understood by reference to the following detailed description when considered in connection with the accompanying drawings wherein:

FIG. 1 is a frontal view of the present invention as it would be seen while in use. The main, curved body of the gasket is shown prior to end user modification to seal to the individual's face. External gasket is stippled grey. Filtration material is denoted by vertical parallel lines. Center attachment clip in its preferred embodiment is visible on midline.

FIG. 2 is a back view of the present invention as it would be seen by the user. The main, curved body of the gasket is shown prior to end user modification to seal to the individual's face. The central attachment portion is shown here in its preferred embodiment as a clip, but is not limited to this form. Filtration material is denoted by vertical parallel lines. Internal gasket denoted by crosshatching.

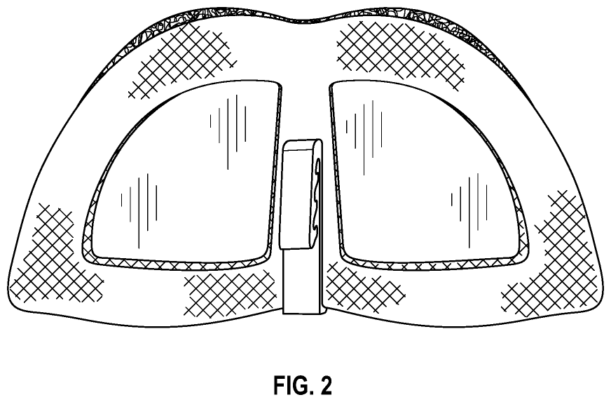

FIG. 3 is a side/profile view of the present invention. The main, curved body of the gasket is shown prior to end user modification to seal to the individual's face. The layered material (seal, filtration material, gasket) is shown in its preferred embodiment, however the invention is not limited to this combination. External gasket is stippled grey. Filtration material is denoted by vertical parallel lines. Internal gasket denoted by crosshatching. A human hand is shown for demonstrative purposes only to illustrate how the invention in its current embodiment may be grasped.

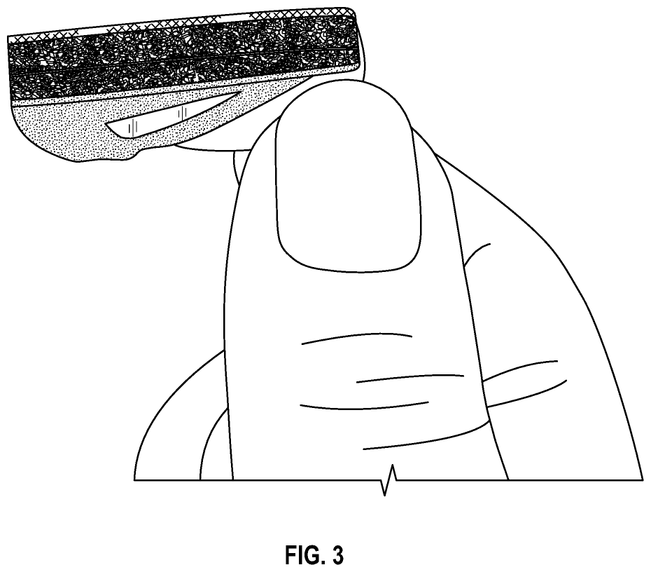

FIG. 4 is an oblique, frontal/top view of the present invention as it would be seen while in use. The main, curved body of the gasket is illustrated in this view to be modified to the end user to form a close seal around the face. External gasket is stippled grey. Filtration material is denoted by vertical parallel lines. Center attachment clip in its preferred embodiment is visible on midline.

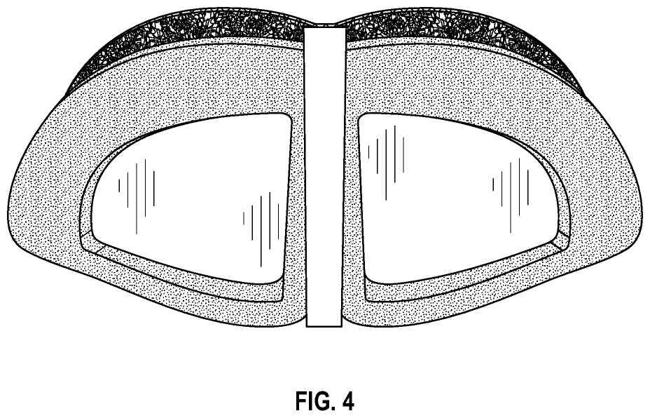

FIG. 5 is an obliqued side view of the present invention. The central attachment portion is shown here in its preferred embodiment as a clip, but is not limited to this form. The layered material (seal, filtration material, gasket) is shown in its preferred embodiment, however the invention is not limited to this combination. External gasket is stippled grey. Filtration material is denoted by vertical parallel lines. Internal gasket denoted by crosshatching.

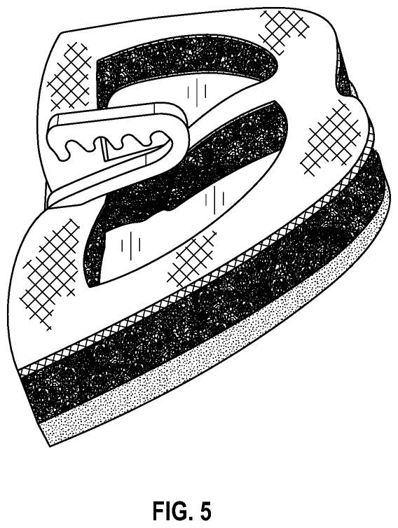

FIG. 6 is a side or profile, exploded view, of the invention in its current embodiment as it would be while in use and properly adjusted by the end user. This allows the examiner to better visualize the invention in its current embodiment with layers and internal structures visible. The malleable internal skeleton which is in the current invention is shown in this view between the filtration layer and internal gasket. External gasket is stippled grey. Filtration material is denoted by vertical parallel lines. Internal gasket denoted by crosshatching. Device is shown in its current, preferred embodiment however it is not limited to this specific combination.

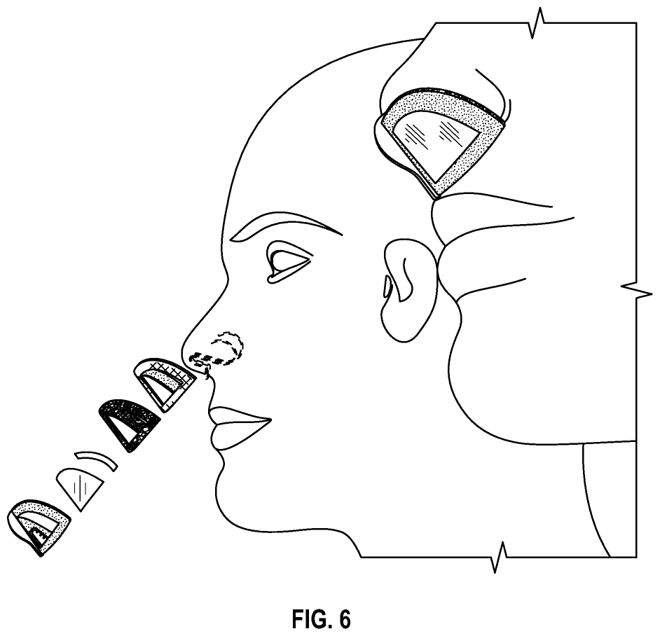

The foregoing are examples only and indicate the wide degree of variability available for manufacturing different sizes or shapes of the apparatus, fitting adjustments to the users' anatomy, and/or allowing the end user to make adjustments.

It should be appreciated that the nasal air filtration apparatus as described in this disclosure may be formed in various ways and of various materials by those skilled in the art. For example, the gasket structure may be a smooth arc, a flattened “M” shape, or have changes within the corner structures to better accommodate different nasal anatomy. The length to width ratio may vary slightly in order to account for differences in nasal protrusion among users. The apparatus may be constructed such that the materials are permanently fused at the edges, or alternately such that the filter material may be replaced with clean material after use.

Examples of materials which may be used to form the parts which form the apparatus include, but are not limited to, biosafe foams, biosafe silicone, or types of plastic for the sealing portion of the gasket; metals or other rigid but malleable material for the internal portion of the gasket; cotton, paper, polypropylene, meltblown plastic, or fiberglass for the filtration portion; and various types of stiff plastics or metals for the clip attachment. The device may be coated with an antimicrobial or static inducing material in order to improve filtration. Alternatively, various other treatments such as essential oils, repository/long acting medications, or decorative elements may be applied to the apparatus.

Various terms have been used within this enclosure to further the understanding of the structure, use, and function of the nasal air filter apparatus. Here, “nares” refers to nostrils, “inspiration” refers to the act of inhaling air, “filtration” refers to the act of removing unwanted particulates from the air, “malleable” refers to material which can be bent with a slight amount of pressure and then retains its' shape.

What is claimed is:

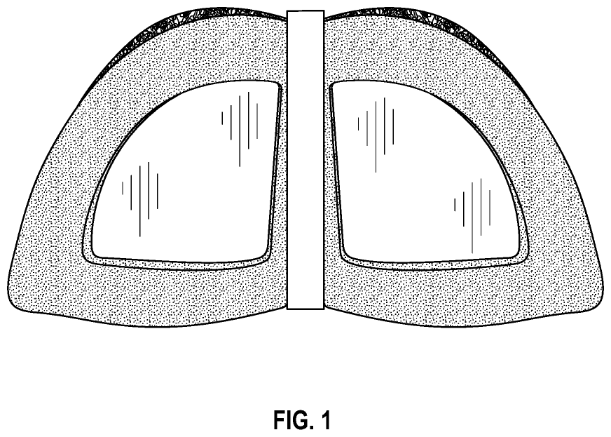

1. A device for filtering air moving through the nares as described in FIG. 1.

2. A device for filtering air moving through the nares, comprising:
a gasket portion to prevent air leakage;
a filtering material;
a malleable internal structure within the gasket to allow individual users to fit/seal device to their anatomy; and
a method to attach the apparatus to a nasal dilator (refer to U.S. provisional patent No. 62/890,961) characterized in that the device is symmetrical from side to side, the attachment method is on midline and the seal is immediately adjacent to wearers' face.

3. A device for filtering air moving through the nares according to claim 2, wherein the gasket portion is composed in part or entirely of silicone, neoprene, latex, or any other type of flexible material to allow for an airtight seal around the wearer's nares.

4. A device for filtering air moving through the nares according to claim 3, wherein the gasket portion also has a decorative aspect or function.

5. A device for filtering air moving through the nares according to claim 2, wherein the filtering material is composed of one or multiple layers of material including, but not limited to, carbon fiber, paper, foam, cotton, fiberglass, and polyester.

6. A device for filtering air moving through the nares according to claim 2, wherein the internal structure of the gasket portion is composed round or flat metal of varying malleability including, but not limited to, copper, stainless steel, zinc, or other composites/alloys.

7. A device for filtering air moving through the nares according to claim 2, wherein the method of attachment to (refer to provisional patent) is composed of, but not limited to, rigid plastic, metal, wood, or other composite material.

8. A device for filtering air moving through the nares according to claim 5, wherein the filtering material has additives including, but not limited to, essential oils, colloidal or particulate silver, antimicrobial medications, pain medications, or activated charcoal.

9. A device for filtering air moving through the nares according to claim 8, wherein the filtering material also has a decorative aspect or function.

10. A device for filtering air moving through the nares according to claim 7, wherein the method of attachment contains additional materials to aid in securing the apparatus such as, but not limited to, magnets, adhesive, or static based attractants.

11. A device for filtering air moving through the nares according to claim 7, wherein the method of attachment is in the form of a hook or clip structure.
